(19) United States
(12) Patent Application Publication
(10) Pub. No.: US 2022/0362784 A1
(43) Pub. Date: Nov. 17, 2022

(54) AIR DISINFECTION DEVICE

(71) Applicant: Shanghai Emperor of Cleaning Hi-Tech Co., LTD, Shanghai (CN)

(72) Inventor: Wei WANG, Shanghai (CN)

(73) Assignee: Shanghai Emperor of Cleaning Hi-Tech Co., LTD, Shanghai (CN)

(21) Appl. No.: 17/489,802

(22) Filed: Sep. 30, 2021

(30) Foreign Application Priority Data
May 12, 2021 (CN) 202121014067.2

(51) Int. Cl. B03C 3/38; B03C 3/04; B03C 3/66; A61L 9/18

(52) U.S. Cl. CPC B03C 3/38; B03C 3/04; B03C 3/66; A61L 9/18

(57) ABSTRACT

An air disinfection device includes a shell having entry and exhaust ports, a power supply installed above the shell, and a dust collecting cylinder installed on the shell, connected to a negative electrode of the power supply, and having air inlet and outlet. The air inlet and the air outlet are communicated with the entry port and the exhaust port, respectively. The conductive rod has one end installed above the shell and connected to a positive electrode of the power supply, and the other end located in the dust collecting cylinder and close to the air inlet. An arrangement direction of the conductive rod is consistent with an axis direction of the dust collecting cylinder. The power supply provides high-voltage current to the conductive rod to form a symmetrically distributed high-voltage electrostatic field inside the dust collecting cylinder to sterilize and disinfect air passing through the dust collecting cylinder.

### CROSS-REFERENCE TO RELATED APPLICATION

This application claims the priority benefit of China application serial no. 202121014067.2, filed on May 12, 2021. The entirety of the above-mentioned patent application is hereby incorporated by reference herein and made a part of this specification.

### BACKGROUND

### Technical Field

The present invention relates to the technical field of air disinfection, in particular to an air disinfection device.

### Description of Related Art

The corona-virus has brought huge pain and loss to human beings. Since the main transmission mode of the corona-virus is air transmission, an effective means is needed to realize automatic indoor air disinfection in a manned environment.

At present, electrostatic technology has been used for indoor air disinfection and dust removal. The existing electrostatic technology uses a two-stage flat-plate high-voltage electrostatic technology, which belongs to a rod-plate structure electric field, that is, a rod electrode is set between two flat electrodes. Due to the formation of a non-closed electric field, some of the electric field lines radiate outward from the opening between the two plates. The electric field force in this area is not enough to kill microorganisms, which makes it impossible to sterilize this area. Therefore, there is a working blind zone in the electric field of the rod-plate structure, and the disinfection effect is not good, and it cannot meet the relevant national requirements, that is, the requirement of 500 cfu/m3. Moreover, the electric field distribution of the electric field of the rod-plate structure is asymmetrical and uneven, which is prone to electric field breakdown and continuous crackling noise, so it is not suitable for a manned environment.

### SUMMARY

The purpose of the present invention is to overcome the existing technical deficiencies and provide an air disinfection device to solve the problems of poor disinfection effect and high noise of the existing high-voltage electrostatic air disinfection technology.

In order to achieve the above objectives, the present invention provides an air disinfection device, which includes a shell, a power supply, a dust collecting cylinder and a conductive rod. The shell has an entry port and an exhaust port. The power supply is installed above the shell and capable of providing high-voltage current. The dust collecting cylinder is installed on the shell and connected to a negative electrode of the power supply. The dust collecting cylinder has an air inlet and an air outlet that are arranged oppositely. The air inlet is communicated with the entry port, and the air outlet is communicated with the exhaust port. One end of the conductive rod is installed above the shell and connected to the positive electrode of the power supply, and the other end of the conductive rod is located in the dust collecting cylinder and close to the air inlet. An arrangement direction of the conductive rod is consistent with a direction of an axis of the dust collecting cylinder, and the high-voltage current is supplied to the conductive rod through the power supply, so that a symmetrically distributed high-voltage electrostatic electric field is formed inside the dust collecting cylinder to sterilize and disinfect air passing through the dust collecting cylinder.

The present invention provides high-voltage current to the conductive rod through the power supply. The electric field lines inside the dust collection cylinder are closed from the conductive rod to the inner wall of the dust collection cylinder, so a closed high-voltage electrostatic electric field is formed. The entire area of the dust collection cylinder is a high-voltage electric field. There is no working blind zone, and the efficiency of sterilization and disinfection is higher. The microorganisms and dust in the air passing through the dust collecting cylinder are charged by the high-voltage electrostatic electric field, and the microorganisms are killed by the electric breakdown effect, and the microorganisms and dust are attached to the dust collecting cylinder under the action of the electric field force. On the inner wall of the cylinder, the air can be sterilized and disinfected and dust can be removed. Since the conductive rod is located on the axis of the dust collecting cylinder, the electric field lines around the conductive rod are symmetrically distributed, and a stable and symmetrical high-voltage electrostatic field is formed in the entire dust collecting cylinder. This avoids the problem of electric field breakdown due to asymmetric electric field distribution, and is noiseless and safer.

According to an embodiment of the present invention, the end of the conductive rod close to the air inlet is pointed.

According to an embodiment of the present invention, a distance from the end of the conductive rod close to the air inlet to the air inlet is 1 mm.

According to an embodiment of the present invention, the air disinfection device further includes a mounting frame mounted above the shell and adjacent to the air outlet, and one end of the conductive rod is mounted above the shell through the mounting frame.

According to an embodiment of the present invention, the air disinfection device further comprises a fixed plate installed on the entry port, the fixed plate is provided with mounting hole, and the dust collecting cylinder is installed at the mounting hole and the air inlet is communicated with the mounting hole.

According to an embodiment of the present invention, a cross section of the dust collecting cylinder is polygonal.

According to an embodiment of the present invention, a cross section of the dust collecting cylinder is circular.

According to an embodiment of the present invention, there are several dust collecting cylinders.

### BRIEF DESCRIPTION OF THE DRAWINGS

FIG. 1 is a perspective view of a circular cross-section of a dust collecting cylinder of an air disinfection device of the present invention.

FIG. 2 is a front view of the dust collecting cylinder of the air disinfection device of the present invention in a circular shape in cross section.

FIG. 3 is a top view of the dust collecting cylinder of the air disinfection device of the present invention in a circular shape in cross section.

FIG. 4 is a perspective view of the dust collecting cylinder of the air disinfection device of the present invention in a hexagonal cross-section.

FIG. 5 is a front view of a hexagonal cross-section of the dust collecting cylinder of the air disinfection device of the present invention.

FIG. 6 is a top view of the cross section of the dust collecting cylinder of the air disinfection device of the present invention in a hexagonal shape.

FIG. 7 is a perspective view of the dust collecting cylinder of the air disinfection device of the present invention with a square cross-section.

FIG. 8 is a front view of a square cross-section of the dust collecting cylinder of the air disinfection device of the present invention.

FIG. 9 is a top view of the dust collecting cylinder of the air disinfection device of the present invention with a square cross-section.

FIG. 10 is a schematic diagram of an arrangement of a plurality of dust collecting cylinders with circular cross-sections of the air disinfection device of the present invention.

FIG. 11 is another arrangement diagram of a plurality of dust cylinders with circular cross-sections of the air disinfection device of the present invention.

FIG. 12 is a schematic diagram of the arrangement of a plurality of dust collecting cylinders with a hexagonal cross section of the air disinfection device of the present invention.

FIG. 13 is a schematic diagram of the arrangement of a plurality of dust collecting cylinders with quadrangular cross-sections of the air disinfection device of the present invention.

### DESCRIPTION OF THE EMBODIMENTS

In order to make the purpose, technical solutions and advantages of the present invention clearer, the present invention will be further described in detail below with reference to the accompanying diagrams and implementation cases. It should be understood that the specific implementation cases described here are only used to explain the present invention, and are not used to limit the present invention. Based on the implementation cases of the present invention, all other implementation cases obtained by those of ordinary skill in the art without creative work shall fall within the protection scope of the present invention.

The present invention provides an air disinfection device, which is used for air disinfection and dust removal. The present invention includes a shell, a power supply, a dust collecting cylinder and a conductive rod. The power supply provides high-voltage current to the conductive rod. The electric field line inside the dust collecting cylinder is closed from the conductive rod to the inner wall of the dust collecting cylinder, so a closed high-voltage electrostatic electric field is formed. The entire area in the dust collection cylinder is high-voltage electric field, there is no working blind zone, and the efficiency of sterilization and disinfection is higher. The microorganisms and dust in the air passing through the dust collecting cylinder are charged by the high-voltage electrostatic electric field, and the microorganisms are killed by the electric breakdown effect, and the microorganisms and dust are attached to the inner wall of the dust collecting cylinder under the action of the electric field force, so as to realize the sterilization and dust removal in the air. Since the conductive rod is located on the axis of the dust collecting cylinder, the electric field lines around the conductive rod are symmetrically distributed, and a stable and symmetrical high-voltage electrostatic field is formed in the entire dust collecting cylinder. This avoids the problem of electric field breakdown due to asymmetric electric field distribution, and is noiseless and safer. The stable symmetrical electric field distribution effectively controls the production of ozone.

The air disinfection device of the present invention will be described below in conjunction with the accompanying figures.

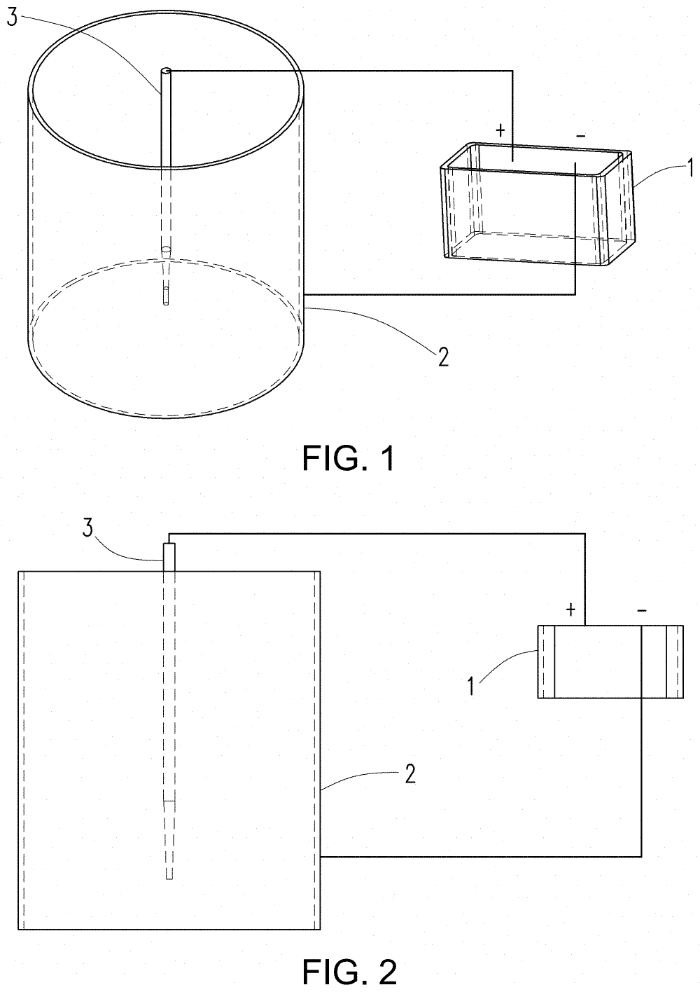

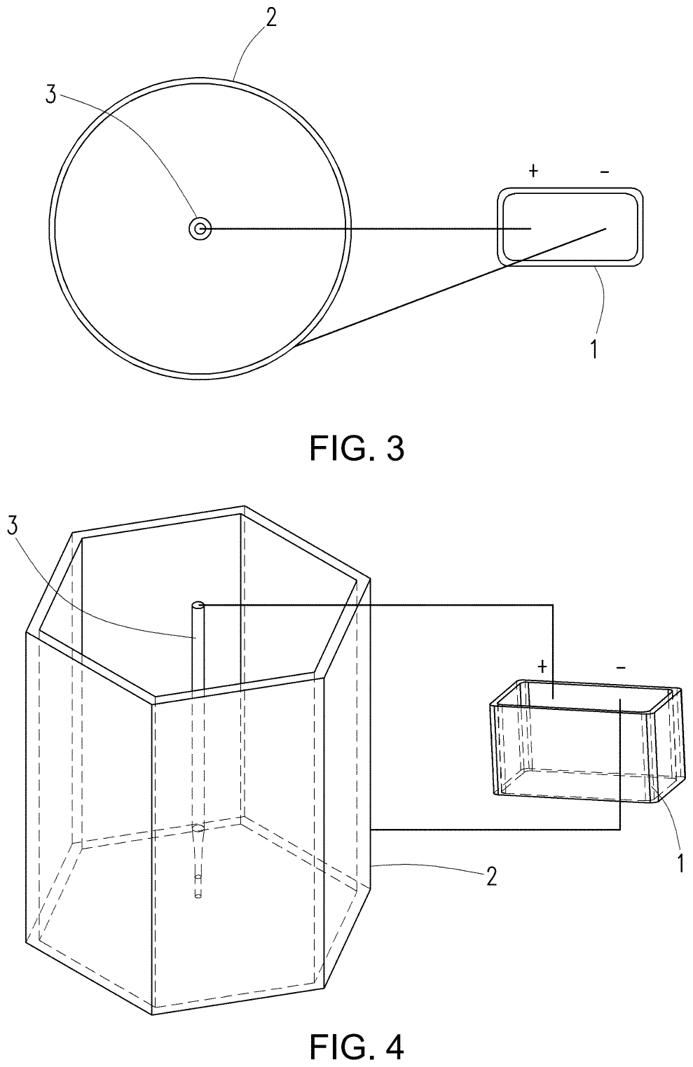

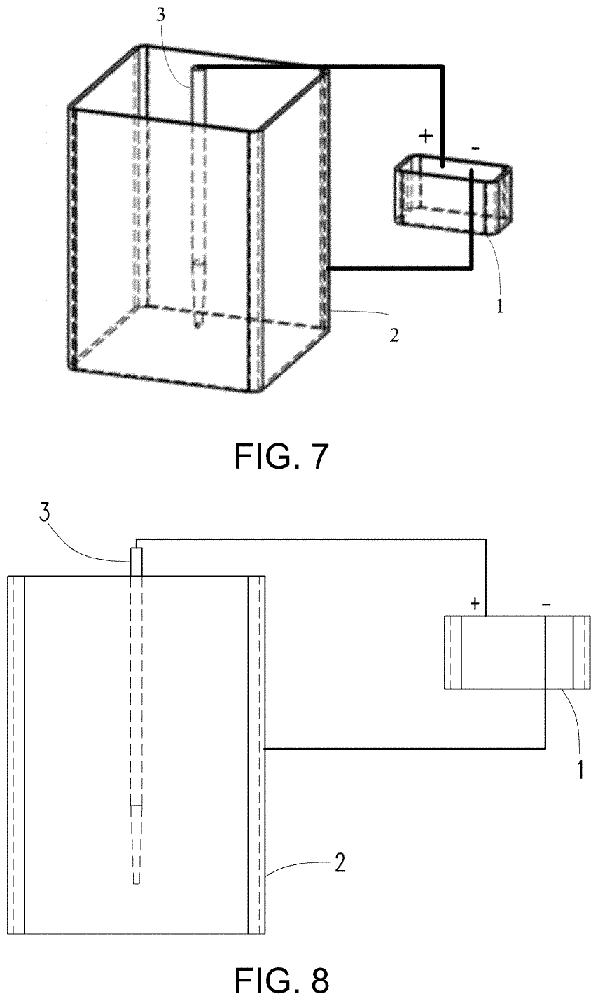

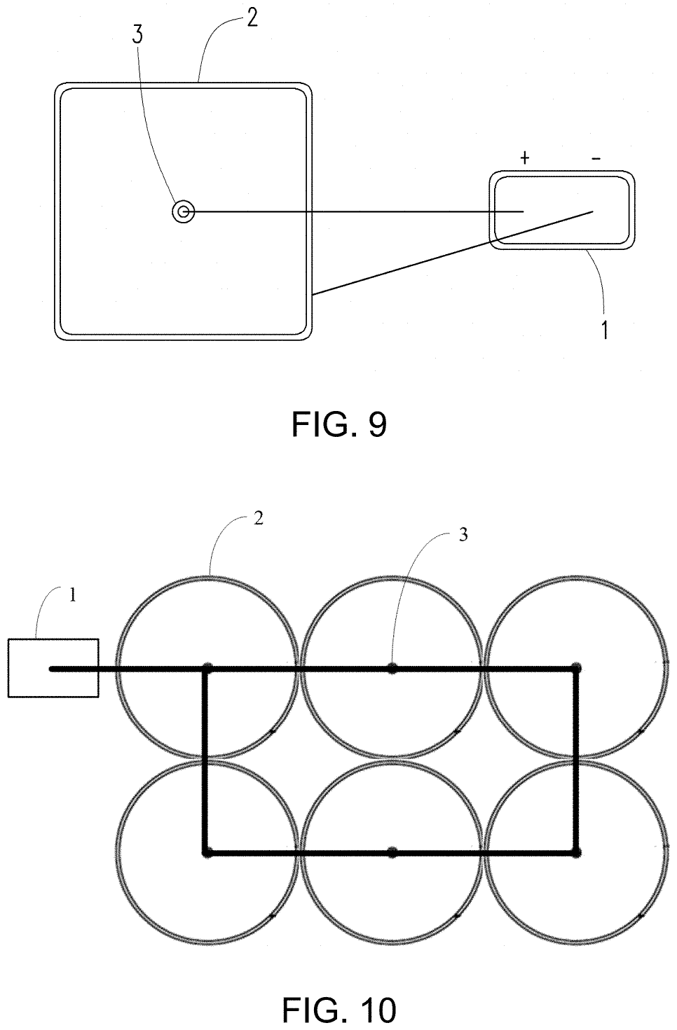

Referring to FIGS. 1, 3-4, 6-7 and 9, in this implementation case, an air disinfection device includes a shell, a power supply 1, a dust collecting cylinder 2 and a conductive rod 3. The shell is provided with an entry port and an exhaust port. The power supply 1 is installed above the shell and can provide high-voltage current. The dust collection cylinder 2 is installed on the shell and connected to the negative electrode of the power supply 1. The dust collection cylinder 2 has opposite air inlets and air outlets. The air inlet is communicated with the entry port, and the air outlet is communicated with the exhaust port. One end of the conductive rod 3 is installed above the shell and connected to the positive electrode of the power supply 1, and the other end of the conductive rod 3 is located in the dust collecting cylinder 2 and close to the air inlet. An arrangement direction of the conductive rod 3 is consistent with the direction of the axis line of the dust collecting cylinder 2, and the power supply 1 provides high-voltage current to the conductive rod 3, so that the inside of the dust collecting cylinder 2 forms a symmetrically distributed high-voltage electrostatic electric field to sterilize the air passing through the dust collecting cylinder 2.

The present invention provides a high-voltage current to the conductive rod 3 through the power supply 1, and the electric field lines inside the dust collecting cylinder 2 are closed from the conductive rod 3 to the inner wall of the dust collecting cylinder 2, so that a closed high-voltage electrostatic electric field is formed. The entire area in the dust collecting cylinder 2 is a high-voltage electric field, there is no blind area, and the efficiency of sterilization and disinfection is higher. The microorganisms and dust in the air passing through the dust collecting cylinder 2 are charged by the high-voltage electrostatic electric field, and the microorganisms are killed by the electric breakdown effect, and the microorganisms and dust are attached to the inner wall of the dust collecting cylinder under the action of the electric field force, so as to realize the sterilization and dust removal in the air. Since the conductive rod 3 is located on the axis of the dust collecting cylinder 2, the electric field lines around the conductive rod 3 are symmetrically distributed, and a stable and symmetrical high-voltage electrostatic field is formed in the entire dust collecting cylinder 2. This avoids the problem of electric field breakdown due to asymmetric electric field distribution, and is noiseless and safer. The generation of ozone is related to the voltage and electric field distribution. The uneven electric field distribution of the existing rod-plate structure easily generates excessive ozone. A stable and symmetrical electric field distribution is formed inside the dust collecting cylinder 2 of the present invention, thereby greatly reducing the generation of ozone. On the one hand, the fastener can deliver high-voltage current to the conductive rod 3, and on the other hand, the fastener is used to fix the conductive rod 3 so that the arrangement direction of the conductive rod 3 is consistent with the direction of the axis of the dust collecting cylinder.

In the first implementation case, the air disinfection device is an independent device for disinfecting and dusting the air. The outside air is continuously sucked into the shell through a fan and discharged from the shell through a dust collecting cylinder 2 for disinfecting and dusting, thus realizing the disinfecting and purifying of the outside air.

In the second implementation case, the air disinfection device is an installation component, which can be installed on the air inlet or outlet of the air conditioner shell, or the filter plate of the air purifier. The air disinfection device disinfects and removes dust from the inhaled outside air, and then discharges the air conditioner or air purifier, so that the air conditioner and air purifier have the function of air disinfection and dust removal. While the air conditioner or air purifier is working, it can disinfect and remove dust from the air, which is more energy-saving and environmentally friendly.

In the third implementation case, the air disinfection device is a component of the air conditioner, and the shell is adapted to the air conditioner shell and is used to install on the inside of the air conditioner shell. The air disinfection device disinfects and removes dust from the outside air sucked into the air conditioner, and then discharges the air conditioner, so that the air conditioner has the function of air disinfection and dust removal. While the air conditioner is working, it can realize the functions of disinfection and dust removal of the air, which is more energy-saving and environmentally friendly.

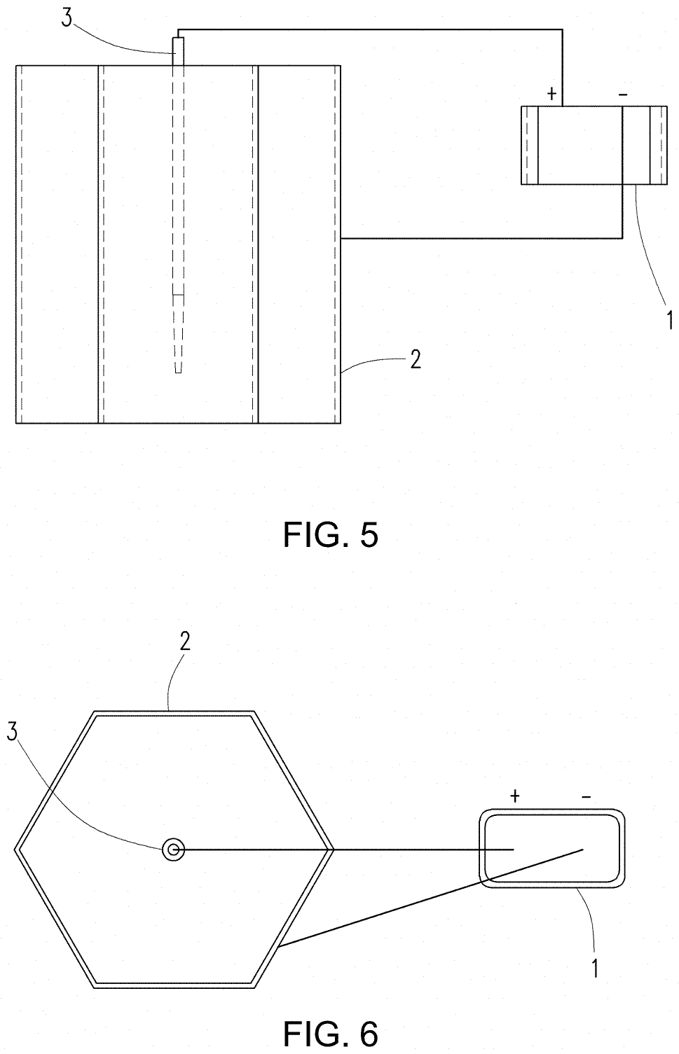

Referring to FIG. 2, FIG. 5, and FIG. 8, further, the end of the conductive rod 3 close to the air inlet is pointed, and the surrounding charge of the pointed part of the conductive rod 3 is particularly dense, and the electric field is particularly strong, thereby forming a strong corona zone. The corona area can achieve a stronger effect of killing microorganisms.

Referring to FIG. 2, FIG. 5, and FIG. 8, furthermore, the distance from the end of the conductive rod 3 close to the air inlet to the air inlet is 1 mm.

Further, the air disinfection device also includes a mounting frame mounted on the shell and adjacent to the air outlet, one end of the conductive rod is installed above the shell through the mounting frame, so that the arrangement direction of the conductive rod 3 is consistent with the direction of the axis line of the dust collecting cylinder 2. When dust collecting cylinder 2 is a plurality of rows, the mounting frame is a square frame structure and includes a plurality of parallel and fixedly connected rods, and one end of the conductive rod 3 is installed and fixed on the corresponding rod.

Furthermore, the air disinfection device further includes a fixing plate attached and installed on the inlet. The fixing plate is provided with a mounting hole, the dust collecting cylinder 2 is installed at the mounting hole, and the air inlet is connected with the mounting hole. When the dust collecting cylinder 2 is in several rows, the mounting holes on the fixing plate are in several rows. The arrangement and distribution of the mounting holes are set according to actual needs to adapt to the required ventilation area, and it's convenient to dissemble and install the single dust collecting cylinder 2, which is more flexible.

Preferably, there are several dust collecting cylinders 2.

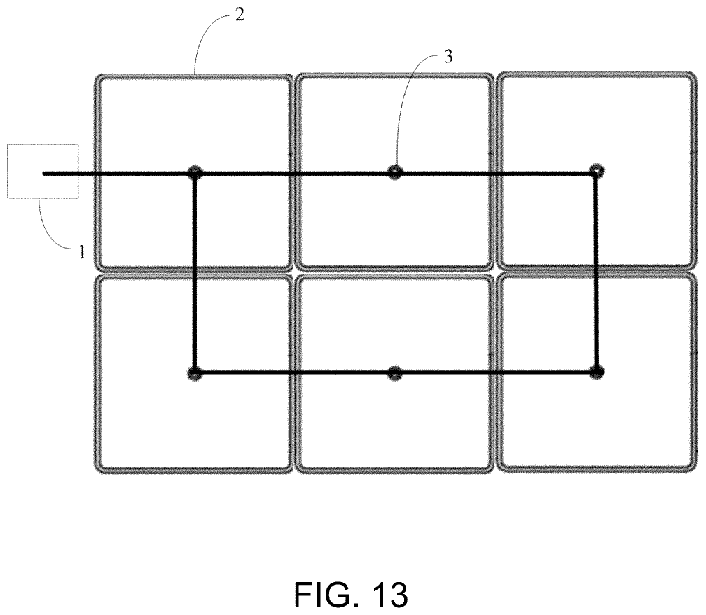

Referring to FIGS. 12 and 13, preferably, the cross section of the dust collecting cylinder 2 is polygonal. When the cross section of the dust collecting cylinder 2 is quadrilateral or hexagonal, the multiple mounting holes on the fixing plate can be aligned and distributed so that the side walls of two adjacent dust collecting cylinders 2 are aligned and connected. In this way, the dust collecting cylinders 2 are arranged tightly and there are no gaps between the dust collecting cylinders 2, the ventilation area is large, and the resistance of the fixed plate to the wind is small, thereby increasing the speed of disinfection. When the cross section of the dust collecting cylinder 2 is a pentagonal shape, a heptagonal shape, or other polygonal shapes, the multiple mounting holes on the fixing plate cannot be aligned and arranged. This makes the area between the mounting holes on the fixing plate obstruct the air circulation, the ventilation area is small, and the wind resistance is larger. However, the distance between the conducting rod 3 in dust collecting cylinder 2 and the inner wall of dust collecting cylinder 2 is not uniform, so the formation inside dust collecting cylinder 2 is only the high-voltage electrostatic field with symmetrical distribution.

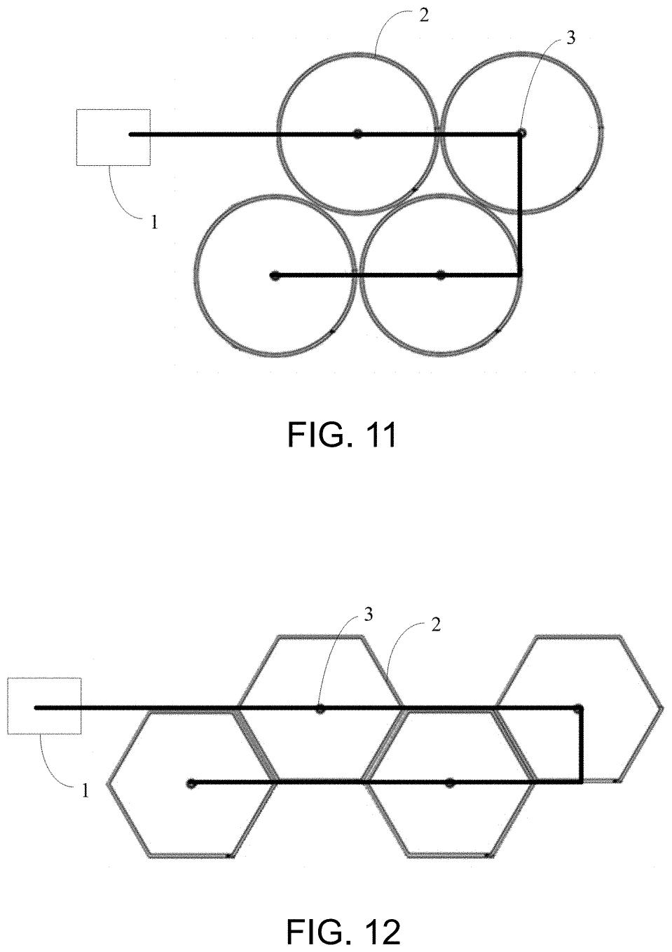

Referring to FIGS. 10 and 11, preferably, the cross section of the dust collecting cylinder 2 is in a circular shape, and the multiple mounting holes on the fixing plate cannot be aligned and arranged. As a result, the area between the mounting holes on the fixing plate will hinder the air circulation, so that the ventilation area is smaller and the wind resistance is larger. The distance between the conductive rod 3 and the inner wall of the dust collecting cylinder 2 in the dust collecting cylinder 2 with a circular cross section is uniform, and a more stable high-voltage electrostatic electric field is formed inside the dust collecting cylinder 2. The high-voltage electrostatic field inside the dust collecting cylinder 2 with a circular cross-section has better stability, better disinfection effect, and less ozone than the high-voltage electrostatic field inside the dust collecting cylinder 2 with a polygonal cross-section.

In this implementation case, the power supply 1 is a DC power supply.

Further, the voltage of the power supply 1 is 4 kv-8 kv.

Preferably, the conductive rod 3 is made of alloy, which is easy to conduct electricity.

Preferably, the dust collecting cylinder 2 is made of metal, which is easy to conduct electricity.

The working process of the air disinfection device of the present invention will be described below.

Under the action of the fan, outside air enters the air inlet of the dust collecting cylinder 2 from the entry port of the shell, and the conductive rod 3 forms a symmetrically distributed high-voltage electrostatic field in the dust collecting cylinder 2. The high-voltage electrostatic electric field makes the microorganisms and dust in the air passing through the dust collecting cylinder 2 be charged, and the microorganisms are killed by the electric breakdown effect, and the microorganisms and dust are attached to the dust collecting cylinder 2 under the action of the electric field force. After sterilization and dust removal, the air flows from the air outlet to the exhaust port, and then from the exhaust port to the outside air, so as to realize the sterilization and dust removal of the air.

By adopting the above technical scheme, the present invention has the following beneficial effects.

The present invention provides a high-voltage current to the conductive rod through the power supply. The inside of the dust collecting cylinder forms a closed high-voltage electrostatic electric field. The entire area inside the dust collecting cylinder is a high-voltage electric field. There is no working blind area, and the efficiency of sterilization and disinfection is higher. The electric field makes the microorganisms and dust in the air passing through the dust collecting cylinder charged, and the microorganisms are killed by the electric breakdown effect, and the microorganisms and dust are attached to the inner wall of the dust collecting cylinder under the action of the electric field force, thereby sterilize and remove dust from the air. Since the conductive rod is located on the axis of the dust collecting cylinder, a stable and symmetrical high-voltage electrostatic electric field is formed inside the whole dust collecting cylinder, which avoids the problem of electric field breakdown due to asymmetric electric field distribution and is noiseless and much safer. The present invention forms a stable and symmetrical electric field distribution inside the dust collecting cylinder, thus greatly reducing the generation of ozone. The dust collecting cylinder with hexagonal and square cross section has lower wind resistance, and the high-voltage electrostatic field formed inside the dust collecting cylinder with circular cross section is more stable and produces less ozone. The wind resistance of this invention is small, and the wind resistance is less than 25 Pa when the air flow is normal. The air disinfection has no noise when working, low power consumption, high disinfection efficiency, and virus killing rate >99%. The amount of ozone released is effectively controlled, and is less than 0.01 mg/m3, which is much lower than the national standard. The present invention provides simple structure, no mechanical movement, sturdiness and durability, long service life, no consumables during use, convenient installation and simple maintenance.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. An air disinfection device, comprising:
a shell, having an entry port and an exhaust port;
a power supply, installed above the shell and capable of providing high-voltage current;
a dust collecting cylinder, installed on the shell and connected to a negative electrode of the power supply, the dust collecting cylinder has an air inlet and an air outlet that are arranged oppositely, the air inlet is communicated with the entry port of the shell, and the air outlet is communicated with the exhaust outlet; and
a conductive rod, wherein one end of the conductive rod is installed above the shell and connected to a positive electrode of the power supply, and the other end of the conductive rod is located in the dust collecting cylinder and close to the air inlet, an arrangement direction of the conductive rod is consistent with a direction of an axis of the dust collecting cylinder, and the high-voltage current is supplied to the conductive rod through the power supply, so that a symmetrically distributed high-voltage electrostatic electric field is formed inside the dust collecting cylinder to sterilize and disinfect air passing through the dust collecting cylinder.

2. The air disinfection device according to claim 1, wherein the end of the conductive rod close to the air inlet is pointed.

3. The air disinfection device according to claim 1, wherein a distance from the end of the conductive rod close to the air inlet to the air inlet is 1 mm.

4. The air disinfection device according to claim 1, further comprising a mounting frame mounted above the shell and adjacent to the air outlet, and one end of the conductive rod is installed above the shell through the mounting frame.

5. The air disinfection device according to claim 1, further comprising a fixed plate installed on the entry port, wherein the fixed plate is provided with a mounting hole, and the dust collecting cylinder is installed at the mounting hole and the air inlet is communicated with the mounting hole.

6. The air disinfection device according to claim 1, wherein a cross section of the dust collecting cylinder is polygonal.

7. The air disinfection device according to claim 1, wherein a cross section of the dust collecting cylinder is circular.

8. The air disinfection device according to claim 1, wherein there are several dust collecting cylinders.
